(19) United States
(12) Patent Application Publication
(10) Pub. No.: US 2017/0296037 A1
(43) Pub. Date: Oct. 19, 2017

(54) ENDOSCOPE APPARATUS

(71) Applicant: OLYMPUS CORPORATION, Tokyo (JP)

(72) Inventor: Masahiro YOSHINO, Tokyo (JP)

(73) Assignee: OLYMPUS CORPORATION, Tokyo (JP)

(21) Appl. No.: 15/098,416

(22) Filed: Apr. 14, 2016

(51) Int. Cl. A61B 1/00; A61B 1/07; A61B 1/06; A61B 1/04; A61B 1/005; A61B 1/233

(52) U.S. Cl. CPC A61B 1/00096; A61B 1/233; A61B 1/07; A61B 1/0676; A61B 1/0646; A61B 1/00006; A61B 1/04; A61B 1/005; A61B 1/00009; A61B 1/0638

(57) ABSTRACT

An endoscope apparatus includes: an endoscope including an insertion portion with flexibility inserted into paranasal sinus of a subject and capable of emitting illuminating light from a distal end of the insertion portion toward the subject inside of the paranasal sinus; and an illumination mechanism configured to emit the illuminating light from the endoscope to the subject in a predetermined direction of an irradiation range of the illuminating light in a mode different from other directions.

### BACKGROUND OF THE INVENTION

### 1. Field of the Invention

The present invention relates to an endoscope apparatus including an endoscope configured to emit illuminating light.

### 2. Description of the Related Art

In recent years, an endoscope including an insertion portion inserted into a subject and configured to emit illuminating light from a distal end of the insertion portion to observe an illuminated site has been widely used in a medical field and the like.

A pickup image acquired by the endoscope is displayed as an endoscopic image on a monitor, and in this case, the endoscopic image is displayed in a state that an upper direction of a bending portion or a predetermined direction in an image pickup device is an upper direction of the endoscopic image.

When the endoscope is inserted into the subject, an actual direction (azimuth), such as a vertical direction, of the endoscope in an observation range observed in the subject may be difficult to figure out, and an operation or the like for moving the endoscope toward a site to be observed may be difficult to smoothly perform.

For example, Japanese Patent Application Laid-Open Publication No. 2001-299695 as a first conventional example discloses an endoscope apparatus, wherein projection windows are arranged at two parts of an inclined surface at a distal end of an insertion portion, a light emitting indicator is projected to a surgical site, and the projected light emitting indicator is displayed in an observation image of a rigid endoscope.

Japanese Patent Application Laid-Open Publication No. 2009-279181 as a second conventional example discloses an endoscope, wherein leak light leaked outside from a light guide fiber configured to guide illuminating light enters an indicator light guide fiber, and the indicator light guide fiber is provided along with an image guide fiber.

U.S. Patent Application Publication No. 2009/0187098 as a third conventional example discloses a system, wherein a light emitting tool is inserted into paranasal sinus, and light emitted from the light emitting tool can be observed from outside of a patient to check an insertion position of the light emitting tool.

### SUMMARY OF THE INVENTION

An aspect of the present invention provides an endoscope apparatus including: an endoscope including an insertion portion with flexibility inserted into paranasal sinus of a subject and capable of emitting illuminating light from a distal end of the insertion portion toward the subject; and an illumination mechanism configured to emit the illuminating light from the endoscope to the subject in a predetermined direction of an irradiation range of the illuminating light in a mode different from other directions.

### BRIEF DESCRIPTION OF THE DRAWINGS

FIG. 1 is a diagram showing an entire configuration of an endoscope apparatus according to a first embodiment of the present invention;

FIG. 2 is a diagram showing a configuration of a distal end side of an insertion portion of an endoscope;

FIG. 3 is a diagram showing an example of arrangement of an illuminating window and an observation window on a distal end surface of the insertion portion;

FIG. 4 is a diagram showing an example of transmission characteristics in a region not provided with a filter and a region provided with a filter;

FIG. 5 is a diagram showing an observation range along with an irradiation range in which illuminating light emitted from the endoscope is applied;

FIG. 6 is an explanatory diagram of an action of the first embodiment;

FIG. 7 is a diagram showing a configuration of the distal end side of the insertion portion when light guiding characteristics of part of a light guide are different from other part;

FIG. 8 is a diagram showing an entire configuration of an endoscope apparatus according to a second embodiment of the present invention;

FIG. 9 is a diagram showing a configuration of a distal end side of an insertion portion of a scanning endoscope;

FIG. 10 is a cross-sectional view of a line A-A in FIG. 9;

FIG. 11 is a diagram showing a waveform of a drive signal for driving piezoelectric elements forming an actuator in a Y axis direction;

FIG. 12 is a diagram showing a spiral trajectory depicted by a distal end of an optical fiber when the actuator is driven by the drive signal;

FIG. 13 is a flowchart showing a process of the second embodiment;

FIG. 14 is a diagram showing a situation of an irradiation range in which illuminating light is applied through a filter region and a non-filter region;

FIG. 15A is a diagram showing a waveform and the like of a drive signal for drive in the Y axis direction;

FIG. 15B is a diagram showing a timing of generating R light corresponding to FIG. 15A;

FIG. 15C is a diagram showing an irradiation range of the illuminating light corresponding to FIG. 15B;

FIG. 16 is a diagram showing an example of a first illumination period and a second illumination period;

FIG. 17A is a diagram showing the drive signal and the illuminating light in the first illumination period and the second illumination period;

FIG. 17B is a diagram showing the illuminating light generated in the second illumination period; and

FIG. 18 is a diagram showing an entire configuration of an endoscope apparatus according to a modification of the second embodiment.

### DETAILED DESCRIPTION OF THE PREFERRED EMBODIMENTS

Hereinafter, embodiments of the present invention will be described with reference to the drawings.

### First Embodiment

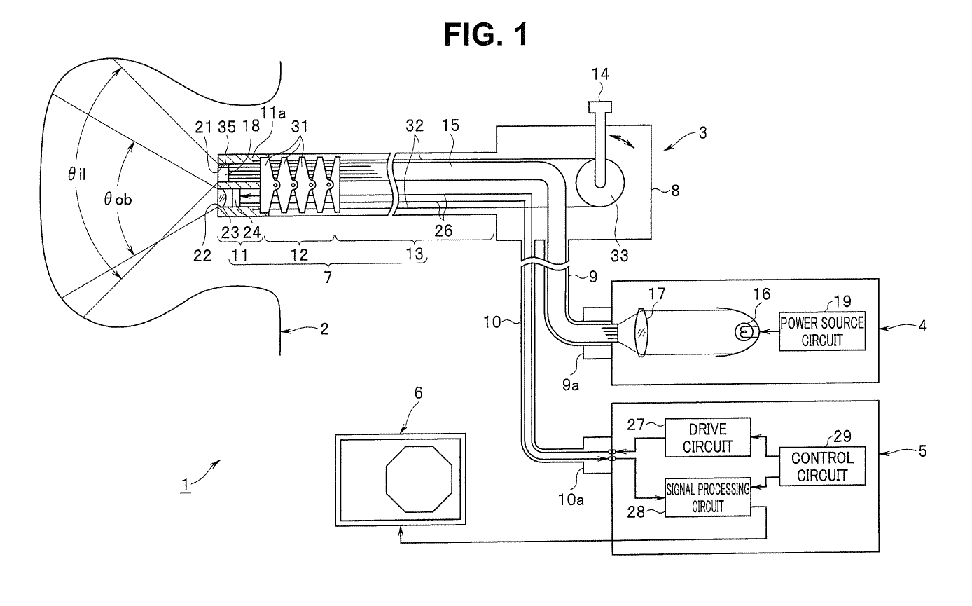

As shown in FIG. 1, an endoscope apparatus 1 according to a first embodiment of the present invention includes: an endoscope 3 inserted into a patient 2 as a subject; a light source apparatus (or a light source unit or a light source section) 4 configured to supply illuminating light to the endoscope 3; a video processor 5 configured to execute signal processing for an image pickup device mounted (included) on the endoscope 3; and a monitor 6 configured to display an endoscopic image.

Note that although the light source apparatus 4 and the video processor 5 that is an image processing apparatus (or an image processing section) configured to execute signal processing are separate components in FIG. 1, the light source apparatus 4 and the video processor 5, or the light source section and the image processing section, may be included in one housing.

The endoscope 3 is inserted into the patient 2 and includes: an insertion portion 7 with flexibility; an operation portion 8 provided at a proximal end of the insertion portion 7; and a light guide cable 9 and a signal cable 10 extended from the operation portion 8.

A light source connector 9a on an end portion of the light guide cable 9 and a signal connector 10a on an end portion of the signal cable 10 are detachably connected to the light source apparatus 4 forming an illumination mechanism and the video processor 5 as an image processing apparatus, respectively.

Note that the illumination mechanism in the present embodiment effectively functions in inspecting or treating inside (surface of the inside) of a site to be inspected (or organ), such as paranasal sinus 2a near a surface layer at a small depth from the surface (for example, the depth from the surface is within about 5 cm) in the patient 2. Specifically, when the illumination light of the illumination mechanism illuminates an irradiation range on the surface inside of the patient 2, the illumination mechanism effectively functions in inspecting or treating the inside of the patient 2 (inside of the paranasal sinus 2a or the like) near the surface layer in the patient 2 so as to allow visually checking a contour of the irradiation range from outside of the patient 2. More specifically, the illumination mechanism effectively functions when illumination of part of a region different from illumination of other regions in the irradiation range allows visually checking or recognizing, from the outside of the patient 2, a predetermined direction or a predetermined azimuth of the part of the region based on a contour of the irradiation range.

Note that as described later, an observation range observed by the endoscope 3 is formed inside of (or part of) the irradiation range, and a predetermined direction in the observation range can also be figured out from the predetermined direction in the irradiation range. The insertion portion 7 includes: a rigid distal end portion 11 provided at a distal end; a bending portion 12 provided adjacent to a proximal end (back end) of the distal end portion 11; and a flexible tube portion 13 with flexibility extended from a proximal end (back end) of the bending portion 12 to a front end of the operation portion 8. The operation portion 8 is provided with a bending operation lever 14 for performing bending operation of the bending portion 12 in arbitrary vertical and horizontal directions.

A light guide fiber (bundle) 15 forming a light guiding portion configured to guide (or transmit) illuminating light is inserted into the insertion portion 7, the operation portion 8, and the light guide cable 9 of the endoscope 3, and an end portion on a hand side of the light guide fiber 15 reaches the light source connector 9a.

The light source apparatus 4 forming the illumination mechanism includes: a lamp 16 as a light source configured to generate illuminating light; a condenser lens 17 configured to condense the generated illuminating light and cause the light to enter the end portion that is an incident end of the light guide fiber 15; and a power source circuit 19 configured to cause the lamp 16 to emit light. Note that the light source is not limited to the lamp 16, and a light emitting diode (abbreviated as LED) may also be used.

The light guide fiber 15 guides the illuminating light entered through the condenser lens 17 to an emission end that is a distal end portion of the light guide fiber 15. The illuminating light is emitted from the distal end portion to the inside of the patient 2 through an illumination lens (or an irradiation lens) 18 as an optical member provided to oppose the distal end portion, and the inside is illuminated.

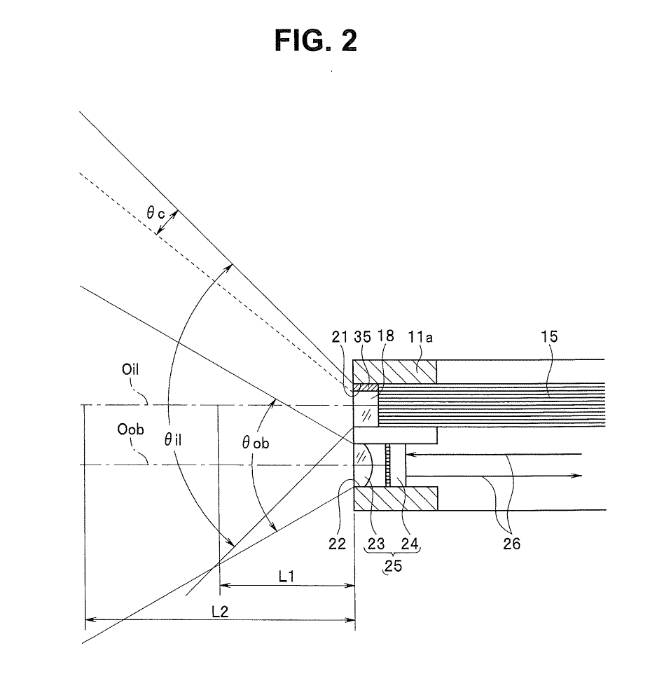

As shown in FIG. 2, distal ends of the illumination lens 18 and the light guide fiber 15 are fixed to an illuminating window 21 (inner surface of the illuminating window 21) of a distal end member 11a forming the distal end portion 11. Note that the bending portion 12 is not illustrated in FIG. 2.

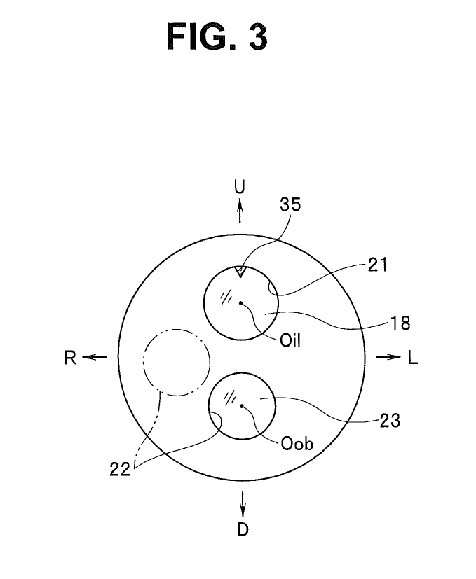

As shown in FIG. 3, an observation window 22 is provided at a position on a lower side of the illuminating window 21 on a distal end surface, and the observation window 22 is provided with: an objective lens 23 as a light receiving element configured to form an optical image; and, for example, a charge coupled device (abbreviated as CCD) 24 as an image pickup device arranged at the image forming position. Note that the position of the observation window 22 is not limited to the position indicated by a solid line in FIG. 2, and the position may be a position as indicated by an alternate long and two short dashes line, for example. In each of FIGS. 1 to 3, each vertical direction on the paper surface coincides with the vertical direction of the distal end portion 11 of the insertion portion 7.

As shown in FIG. 2, the objective lens 23 and the CCD 24 form an image pickup apparatus 25 configured to pick up an image in an observation range equivalent to an incident angle equal to or smaller than an observation view angle bob relative to the object, such as the site to be inspected, that is inside of the patient 2.

An illumination angle θil as an emission angle of the illuminating light is set for the illuminating light emitted from the illuminating window 21 so as to irradiate the irradiation range substantially covering the observation range.

As shown in FIG. 1 or 2, the illumination angle θil for emitting the illuminating light from the illuminating window 21 forming the illumination mechanism is set to an emission angle greater than the observation view angle θob forming an observation field of view.

Note that the irradiation range changes according to a distance from a distal end surface of the distal end portion 11 where the illuminating window 21 is positioned to the object to which the illuminating light is applied. Similarly, the observation range changes according to the distance from the distal end surface of the distal end portion 11 where the observation window 22 is positioned to the object generating reflected light by reflecting the irradiating light. The irradiation range and the observation range will be described later in FIG. 5.

As shown in FIG. 1, the CCD 24 is connected to a distal end of a signal line 26 inserted into the insertion portion 7 and the like, and a back end of the signal line 26 reaches a contact point of a signal connector 10a.

The video processor 5 connected with the signal connector 10a includes: a drive circuit 27 configured to create a drive signal for driving the CCD 24; a signal processing circuit (or an image creation circuit) 28 configured to apply signal processing to an image pickup signal as an output signal outputted from the CCD 24 to create an image signal; and a control circuit 29 configured to control the drive circuit 27 and the signal processing circuit 28.

The image signal created by the signal processing circuit 28 is inputted to the monitor 6, and the monitor 6 displays an image of the image signal as an endoscopic image.

The bending portion 12 in the insertion portion 7 is formed by pivotably connecting a plurality of bending pieces 31 at vertical and horizontal positions in a longitudinal direction (FIG. 1 simply shows a configuration pivotable only in the vertical direction). Bending wires 32 are inserted in the longitudinal direction at positions near vertical and horizontal inner walls in the insertion portion 7 (FIG. 1 simply shows only the bending wires 32 bent in the vertical direction).

Distal ends of the bending wires 32 are fixed to the distal end portion 11 or the bending piece 31 at the distal end, and back ends of the bending wires 32 are wound around a pulley 33 rotatably arranged in the operation portion 8. The bending operation lever 14 is attached to a rotation axis of the pulley 33 (FIG. 1 simply shows only the pulley 33 and the bending operation lever 14 for bending in the vertical direction). An action of turning the bending operation lever 14 can be performed to turn the pulley 33 to pull one of the pair of bending wires 32 to bend the bending portion 12 toward the side that the bending wire 32 is pulled.

In the present embodiment, a filter 35 (part indicated by oblique lines with small intervals) with predetermined transmission characteristics is provided at an upper position equivalent to an upper direction that is a predetermined direction of the observation range in the illumination lens 18 (arranged on an optical path of the illuminating light) as shown in FIGS. 2 and 3. Note that the upper, lower, left, and right directions of the bending portion 12 are indicated by U, D, L, and R in FIG. 3. FIG. 3 shows a case of the distal end surface (of the insertion portion 7) as viewed from a front side of the distal end surface, and the horizontal direction is switched from a case of the distal end surface as viewed from the proximal end side of the insertion portion 7.

Although the filter 35 has, for example, a wedge shape (triangular shape) in an example shown in FIG. 3 and the like, the shape is not limited to the wedge shape, and the shape may be circular, elliptic, rectangular, or the like. As shown in FIG. 3, the illuminating window 21 is circular, and the illumination lens 18 has characteristics of rotational symmetry about an optical axis Oil of the illumination lens 18. Therefore, the illumination lens 18 emits the illuminating light within the range of the illumination angle θil indicated by a solid line in FIG. 2.

As described, the wedge-shaped filter 35 is provided at an upper position that is an upper direction in the circular illumination lens 18. Therefore, illuminating light (as second illuminating light) for checking the direction that is illuminating light with transmission characteristics different from a part or a region not provided with the filter 35 is emitted to a part or a region provided with the filter 35.

The part or the region provided with the filter 35 will also be called a filter region, and the part or the region not provided with the filter 35 will also be called a non-filter region. Note that although the illumination lens 18 as an optical member includes the filter region and the non-filter region in the present embodiment, an irradiation lens 56 may be an optical member including only a non-filter region in a case of a second embodiment described later.

As shown in FIG. 2, although the illumination lens 18 emits the illuminating light within the range of the illumination angle θil, the illumination lens 18 emits the second illuminating light as illuminating light reflecting the transmission characteristics of the filter region in the filter region provided with the filter 35. In FIG. 2, an emission angle range of the illuminating light based on the filter region is indicated by θc. The filter region is provided only near the upper position in the circular illumination lens 18, and the emission angle range θc of the illuminating light based on the filter region is 0 in other directions.

The non-filter region is used for normal illumination, that is, for illuminating, by illuminating light for illumination (as first illuminating light), a first region (or a first irradiation range) that is a majority of the region (region accounting for at least a half of the area) in the irradiation range covering the observation range to be observed. On the other hand, the filter region is used for illumination that allows visually checking a predetermined direction in the observation range or the irradiation range from the outside of the patient 2, and the region is a second region (or a second irradiation range) excluding the first region in the irradiation range. Therefore, the irradiation range includes the first region (or the first irradiation range) accounting for a majority of the irradiation range and the remaining second region (or the second irradiation range).

In the present embodiment, the light source apparatus 4 configured to generate the illuminating light, the light guide fiber 15 configured to guide the illuminating light, and the illumination lens 18 as an optical member provided with the filter 35 form an illumination mechanism configured to emit illuminating light for facilitating figuring out a predetermined direction in the observation range or the irradiation range. Note that in the present embodiment, it may be defined that the light guide fiber 15 as a light guiding portion configured to guide the illuminating light and the optical member (the illumination lens 18 as an optical member) provided with the filter 35 form the illumination mechanism (in the second embodiment described later, the illumination mechanism also includes a light source unit 71 equivalent to the light source apparatus 4).

In the present embodiment, the predetermined direction in the observation range coincides with the predetermined direction in the illumination range. Therefore, the predetermined direction in the observation range and the predetermined direction in the illumination range can be interchanged (rephrased with each other).

In the present embodiment, the illumination range or the observation range can be approximated to be substantially circular. Therefore, to facilitate figuring out the predetermined direction, the second region to which the second illuminating light is emitted is formed in a predetermined direction, such as an upper direction in a circumferential direction based on a position of a center of the illumination range or the observation range. A position of a center of gravity may be adopted in place of the position of the center, including a case in which the illumination range or the observation range cannot be approximated to be circular.

The predetermined direction is set according to, for example, the upper direction that is a reference in an endoscopic image formed by picking up an image of the observation range (in other words, corresponding to the observation range). A surgeon observes the endoscopic image displayed on the monitor 6 to perform an inspection, a treatment, or the like. Therefore, if the surgeon can figure out (check) the actual direction of the upper direction in the endoscopic image, the surgeon can smoothly and easily perform an operation involving directivity, such as a movement operation of moving the distal end portion 11 to allow observing the site to be inspected or treated. On the other hand, the operation involving the directivity cannot be smoothly performed if the actual direction of the upper direction in the endoscopic image cannot be figured out (checked).

The upper direction in the endoscopic image corresponds to a predetermined direction in an image pickup surface of the CCD 24 arranged on the distal end portion 11, and the upper direction coincides with the bending direction of the bending portion 12 in the upper direction.

Although the predetermined direction corresponds to the upper direction when the endoscopic image is displayed on the monitor 6 in the case described below, the predetermined direction is not limited to the case in which the predetermined direction is set to the upper direction.

As described, the non-filter region functions to illuminate the irradiation range that covers the observation range, like normal illuminating light. On the other hand, the filter region illuminates part of the region of the irradiation range to facilitate optically identifying or distinguishing the illumination from the illumination based on the non-filter region. The illumination allows distinguishing or identifying the direction or the azimuth of part of the region in the irradiation range to identify the upper direction or the azimuth of the endoscopic image equivalent to the direction in which the filter 35 is provided in the distal end portion 11 or equivalent to the upper direction of the CCD 24.

The non-filter region is used for emitting the illuminating light so as to cover the observation range, and a large occupation area in the illumination lens 18 is desirable. On the other hand, the filter region just needs to allow identifying the direction of part of the region of the irradiation range irradiated through the filter region, and a smaller occupation area can be set compared to the non-filter region. For example, the occupation area of the non-filter region in the illumination lens 18 may be set to 90 to 98%, and the occupation area of the filter region may be set to about 10 to 2%.

Therefore, although the irradiation range includes the first irradiation range based on the non-filter region and the second irradiation range based on the filter region, the irradiation range can be approximated to be substantially equal to the first irradiation range based on the non-filter region.

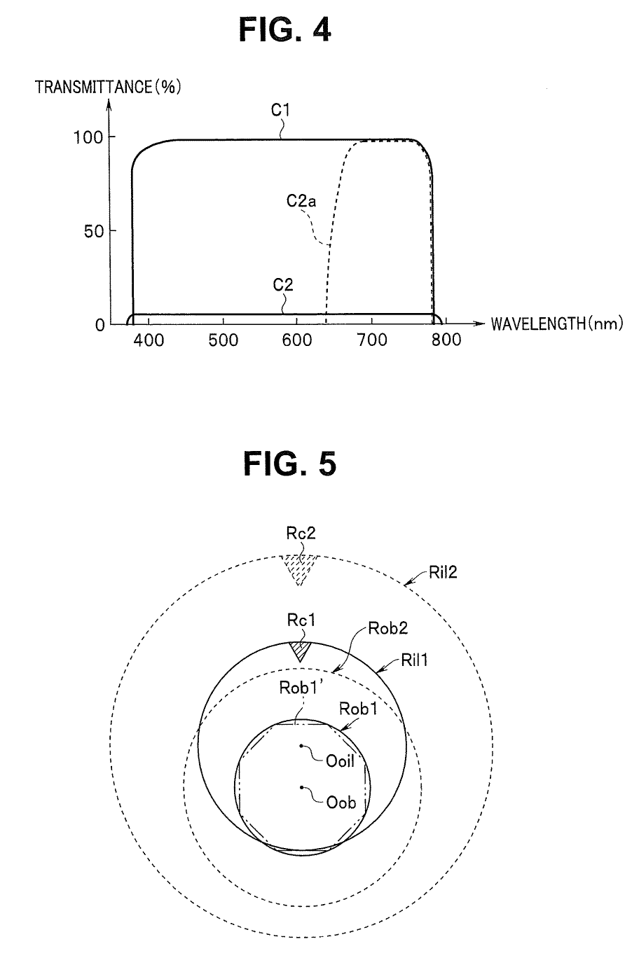

FIG. 4 shows an outline of characteristics of transmittance of the illuminating light emitted from the non-filter region and the non-filter region in the illumination lens 18.

The non-filter region has a transmission characteristic C1 that light of a visible wavelength region (380 nm to 780 nm) generated by the light source apparatus 4 is transmitted with almost no attenuation. On the other hand, the filter region has a transmission characteristic C2 of about 5%, with the light attenuated about 95% throughout the entire visible wavelength region, for example.

Therefore, the illuminating light passing through the non-filter region illuminates the first irradiation range that is the part where the illuminating light is applied, at an illumination intensity in a state with almost no light quantity loss of the illuminating light. On the other hand, the illuminating light passing through the filter region illuminates the second irradiation range that is the part where the illuminating light is applied, at an illumination intensity at which the light is approximately shielded.

In this case, when the irradiation range is viewed from the outside of the patient 2, the direction of the filter region can be optically checked based on the direction of the dark second irradiation range in the irradiation range. In other words, the direction of the dark and invisible second irradiation range can be checked by visually checking only the first irradiation range through the non-filter region.

Note that although FIG. 4 shows an example of the filter region with the transmission characteristic C2 with which the light is almost shielded, the filter region is not limited to the case of the transmission characteristic C2. For example, the filter region may be set to a transmission characteristic C2a with which only part of the wavelength region is transmitted, such as a red wavelength region in the visible region, as indicated by a dotted line.

In this case, the second irradiation range based on the filter region is illuminated with a tone different from the illumination based on the first irradiation range when viewed from the outside of the patient 2, and the direction of the filter region can be optically checked.

The light quantity of the illuminating light based on the filter region is a light quantity at least smaller than a light quantity of the illuminating light in the case of the non-filter region (in both cases of the transmission characteristics C2 and C2a in FIG. 4). Therefore, the illuminating light is emitted with the light quantity of second illuminating light smaller than the light quantity of first illuminating light, wherein the first illuminating light is the illuminating light in the case of the non-filter region, and the second illuminating light is illuminating light based on the filter region.

FIG. 5 shows an outline of a case in which the illuminating light is emitted from the illuminating window 21 provided with the illumination lens 18 to an inner wall surface side inside of the patient 2 on the front side of the illuminating window 21 in FIG. 2, showing irradiation ranges when the inner wall surface is at distances L1 and L2 from the distal end surface and showing observation ranges observed from the observation window 22.

Solid lines in FIG. 5 show an irradiation range Ril1 when the inner wall surface (object) is at a position of the distance L1 from the distal end surface of the distal end portion 11 in FIG. 2 and show an observation range Rob1 in the case.

Dotted lines in FIG. 5 show an irradiation range Ril2 when the inner wall surface is at a position of the distance L2 that is twice the distance L1 from the distal end surface of the distal end portion 11 in FIG. 2 and show an observation range Rob2 in the case.

Centers of the irradiation ranges Ril1 and Ril2 in FIG. 5 are positions on the optical axis Oil of the illumination lens 18, and centers of the observation ranges Rob1 and Rob2 are positions on an optical axis Oob of the objective lens 23. In FIG. 5, the second irradiation ranges based on the filter region are indicated by Rc1 and Rc2. The first irradiation ranges based on the non-filter region are remaining ranges after excluding the second irradiation ranges Rc1 and Rc2 in the irradiation ranges Ril1 and Ril2, respectively.

Note that as for the observation ranges Rob1 and Rob2 illustrated in circular shapes by a solid line and a dotted line in FIG. 5, substantial observation ranges used in the display of an endoscopic image are different from the circular shapes when the image pickup surface of the CCD 24 is, for example, square. In the observation range Rob1 illustrated in a circular shape in FIG. 5 for example, parts of four corners of the square in the image pickup surface are dark. Therefore, the parts are excluded from the observation range, and an octagonal observation range Rob1′ is formed as indicated by an alternate long and two short dashes line.

Even when the observation range is octagonal, the observation range can be approximated to be a circular observation range without directional dependency with respect to an arbitrary radial direction. Note that an observation range with directional dependency may be defined without performing the approximation.

As can be understood from FIGS. 2, 5, and the like, the illumination angle θil defining the irradiation range is set to satisfy a relationship of θil>θob with respect to the observation view angle θob defining the observation range in the present embodiment. As can be understood from FIG. 5, the illumination angle θil and the filter region are set so that the second irradiation range based on the filter region is formed (substantially) outside of the observation range in the present embodiment.

In this way, the illumination angle Oil and the filter region are provided to form the second irradiation range based on the filter region outside of the observation range in the present embodiment. Therefore, the second irradiation range does not affect the observation. For example, if the second irradiation range appears in the observation field of view, an observation function in the observation field of view may be reduced. In the present embodiment, generation of a case in which the observation function is reduced is eliminated.

The endoscope apparatus 1 of the present embodiment includes: the endoscope 3 including the insertion portion 7 with flexibility inserted into the paranasal sinus of the patient 2 forming the subject, the endoscope 3 capable of emitting the illuminating light from the distal end of the insertion portion 7 toward the subject in the paranasal sinus; and the illumination lens 18 provided with (the light source apparatus 4 and) the light guide fiber 15 and the filter 35 forming the illumination mechanism configured to emit the illuminating light from the endoscope 3 to the subject in a predetermined direction in the irradiation range of the illuminating light in a mode different from the other directions.

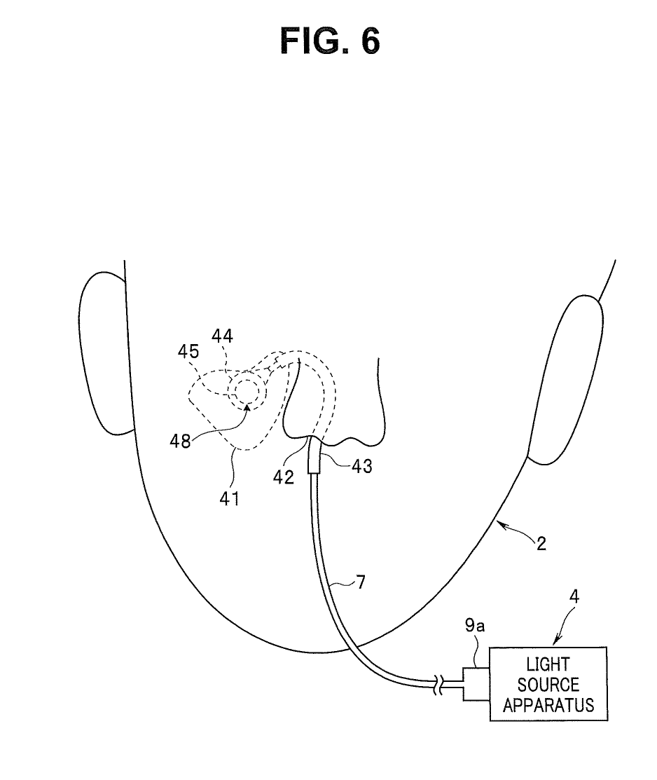

Next, an action (operation) of the present embodiment will be described. FIG. 6 shows an explanatory diagram of a situation in which an inspection is performed by inserting the insertion portion 7 of the endoscope 3 into the paranasal sinus 2a of the patient 2.

To inspect a diseased part or the like inside of, for example, maxillary sinus 41 in the paranasal sinus 2a, the surgeon inserts the insertion portion 7 from nostril 42 through a guide tube 43 as shown in FIG. 6. The guide tube 43 has, for example, a curved shape close to a shape of a hollow path from the nostril 42 to the maxillary sinus 41.

The surgeon inserts a distal end side of the guide tube 43 from the nostril 42 so that the distal end reaches inside of the maxillary sinus 41, and the surgeon inserts the distal end of the insertion portion 7 from an opening of a proximal end of the guide tube 43.

Note that for the surgeon to smoothly perform the operation of inserting the insertion portion 7, an operation of rotating the insertion portion 7 about the longitudinal direction of the insertion portion 7 is often performed. Therefore, the surgeon often cannot figure out the actual direction of the upper direction of the endoscopic image when the insertion operation is performed.

The surgeon further performs an operation of moving the distal end of the insertion portion 7 toward a distal end opening side of the guide tube 43, and the surgeon causes the distal end of the insertion portion 7 to protrude from the distal end opening. FIG. 6 shows this state.

The illuminating light of the light source apparatus 4 is guided by the light guide fiber 15. The guided illuminating light opens through the illumination lens 18 and is emitted toward a sinus inner wall side opposing the illuminating window 21 in the maxillary sinus 41. An irradiation range 44 is formed, in which the illuminating light is applied to the sinus inner wall opposing the illuminating window 21. An observation range 45 that can be observed (image can be picked up) from the observation window 22 is formed inside of the irradiation range 44.

In the irradiation range 44, the filter region forms a second irradiation range (second region) 48 that is an irradiation range almost close to the shielded state compared to the first irradiation range based on the non-filter region. The surgeon can visually recognize the first irradiation range brightly illuminated based on the non-filter region and the second irradiation range 48 close to the shielded state, from the outside of the patient 2. The surgeon can recognize or figure out the direction of the second irradiation range 48 in the irradiation range 44 or the observation range 45.

In FIG. 6, the second irradiation range 48 is in a direction (azimuth) on a lower side of the observation range 45 or the irradiation range 44. Note that the observation range 45 in a state optically unrecognizable from the outside of the patient 2 in FIG. 6 substantially coincides with a display region of the endoscope image displayed on the monitor 6. However, in the endoscopic image displayed on the monitor 6, the image pickup signal picked up on the image pickup surface of the CCD 24 is read at a predetermined timing and displayed as an endoscopic image in an endoscopic image display area of the monitor 6. Therefore, the direction of the endoscopic image display area does not change even if the distal end portion 11 is rotated about the longitudinal direction (the endoscopic image displayed in the endoscopic image display area is rotated).

In this way, the surgeon can figure out the upper direction in the endoscopic image of the observation range 45 or the upper direction of the bending portion 12 based on the direction of the second irradiation range 48 that can be figured out from the outside of the patient 2.

Therefore, even when the surgeon intends to inspect (or observe) a site different from the currently observed observation range 45, the surgeon can figure out the direction in which the distal end portion 11 of the insertion portion 7 needs to be moved to inspect the site, and the surgeon can smoothly inspect an arbitrary site inside of the maxillary sinus 41.

Although the case of inspecting inside of the maxillary sinus 41 is described, a similar effect can be attained in inspecting other sites in the paranasal sinus 2a. The upper direction as a predetermined direction in the endoscopic image of the observation range 45 can also be figured out when a treatment instrument is used to perform a treatment, and this facilitates the treatment in a state that the treatment instrument is put into the observation range.

In the present embodiment, the irradiation mechanism is provided to form the second irradiation range 48 outside of the observation range 45, and this can eliminate a situation that the observation of part of the region of the observation range 45 becomes difficult when the second irradiation range 48 is formed inside of the observation range 45.

In other words, the reduction in the observation function caused by the second irradiation range 48 can be prevented.

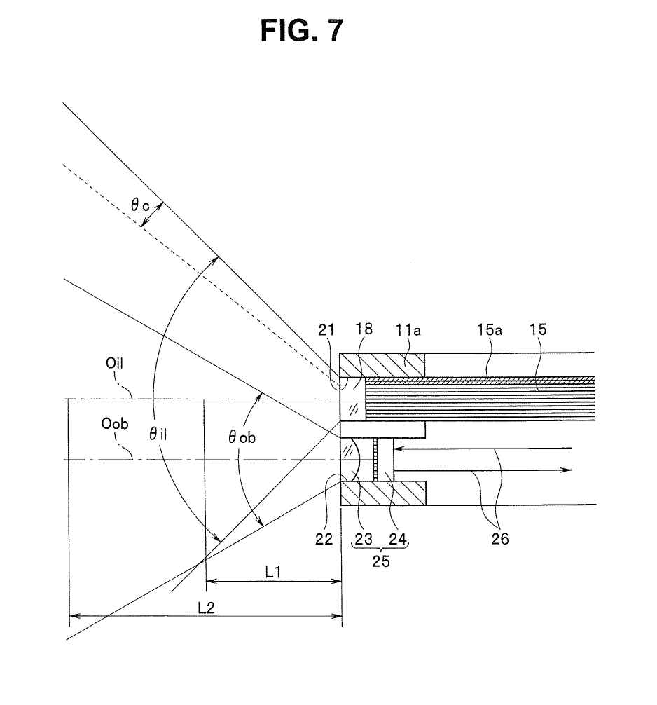

Note that although the illumination mechanism is provided with the filter 35 on the illumination lens 18 as an optical member in the example described above, the case is not limited to this. To provide a function substantially equivalent to the case including the filter 35, a light guiding characteristic of a light guide fiber part (indicated by 15a) as part of the light guide fiber 15 may be set to a characteristic different from light guiding characteristics of other light guide fiber parts as shown for example in FIG. 7. For example, the light guiding characteristic of the light guide fiber part 15a may be set to a characteristic such as a transmission characteristic like the transmission characteristic C2 or C2a in FIG. 4.

When the light guide fiber 15 as in FIG. 7 is used, an effect similar to the case including the filter 35 is attained. Next, the second embodiment of the present invention will be described.

### Second Embodiment

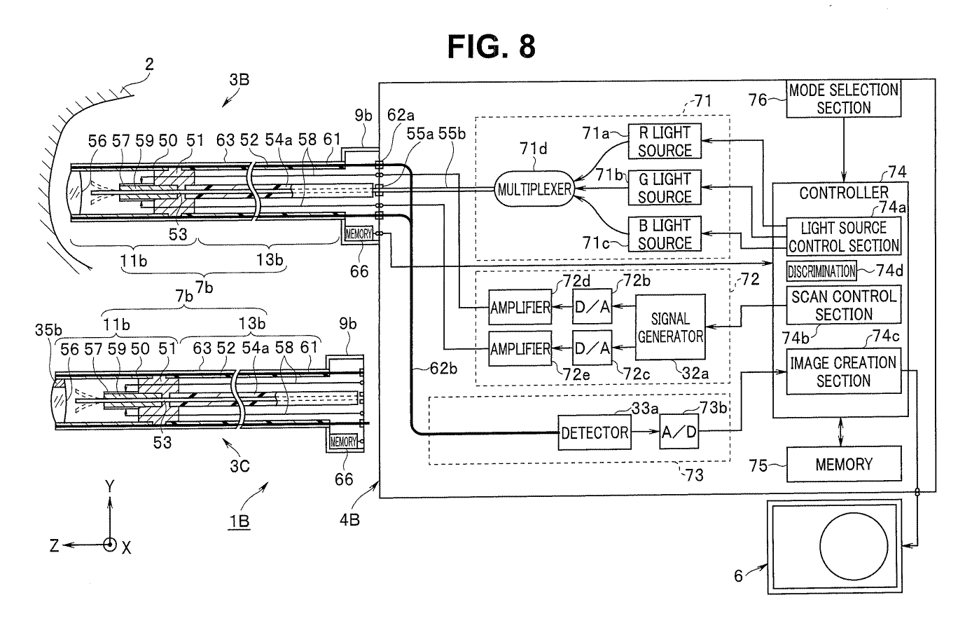

FIG. 8 shows an endoscope apparatus 1B according to the second embodiment of the present invention. The endoscope apparatus 1B shown in FIG. 8 includes: a scanning endoscope 3B configured to two-dimensionally scan illuminating light; an endoscope apparatus body (abbreviated as an apparatus body) 4B detachably connected with the scanning endoscope 3B; and the monitor 6 connected to the apparatus body 4B.

Although the apparatus body 4B according to the present embodiment includes the light source unit 71 configured to generate illuminating light, a controller 74 including an image creation section (or an image processing apparatus) 74c configured to create an image signal, and the like as described later, the light source unit 71 may be a component separate from the image creation section 74c.

The endoscope apparatus 1B includes the scanning endoscope 3B and a scanning endoscope 3C in which only an optical member provided on a distal end portion 11b is different, and the different types of scanning endoscopes 3B and 3C can be selectively connected to the apparatus body 4B. FIG. 8 shows a state in which the scanning endoscope 3B is connected to the apparatus body 4B.

In the present embodiment, for example, the scanning endoscope 3C is provided with a filter 35b on the optical member (the irradiation lens 56 as the optical member) as an illumination mechanism configured to emit the illuminating light to facilitate figuring out the predetermined direction in the observation range or the irradiation range as in the first embodiment.

On the other hand, the scanning endoscope 3B is not provided with the filter 35b on the optical member, and in the present embodiment, an illumination mechanism having a function similar to the case of the scanning endoscope 3C provided with the filter 35b is included in the case of the scanning endoscope 3B.

In other words, assuming that the illumination mechanism in the case of the scanning endoscope 3c including the optical member provided with the filter 35b is a first illumination mechanism, the endoscope apparatus 1B of the present embodiment includes the first illumination mechanism and a second illumination mechanism as an illumination mechanism in the case of the scanning endoscope 3B without the optical member provided with the filter 35b.

The scanning endoscope 3B or 3C includes an insertion portion 7b with flexibility in an elongated shape that can be inserted into the paranasal sinus 2a or the like of the patient 2, and a connector 9b for detachably connecting the scanning endoscope 3B or 3C to the apparatus body 4B is provided at a proximal end (back end) of the insertion portion 7b.

The insertion portion 7b includes the rigid distal end portion 11b and a flexible tube portion 13b with flexibility, extending from a back end of the distal end portion 11b to the connector 9b. Note that a freely bendable bending portion may be provided between the distal end portion 11b and the flexible tube portion 13b, and an operation portion provided with an operation knob or the like for bending the bending portion may be provided between the flexible tube portion 13b and the connector 9b.

The distal end portion 11b includes a cylindrical member 50 as a rigid barrel-shaped member. A distal end of a cylindrical tube 52 with flexibility is connected to a rigid holding member 51 holding a back end of the cylindrical member 50. A back end of the cylindrical tube 52 is fixed to the connector 9b.

An optical fiber 53 forming a light guiding portion or a light guiding member for guiding incident light is inserted into the insertion portion 7b.

A proximal end (back end) of the optical fiber 53 is connected to an optical fiber 55b inside of the apparatus body 4B at an optical connection portion 55a in the connector 9b.

The light generated by the light source unit 71 inside of the apparatus body 4B enters, as incident light, the proximal end of the optical fiber 53 through the optical fiber 55b. The incident light guided by the optical fiber 53 is emitted as illuminating light from a distal end surface of the optical fiber 53. The illuminating light emitted from the distal end surface goes through the condensing lens (or the irradiation lens) 56 as an optical member opposing the distal end surface and attached to an illuminating window at a distal end of the cylindrical member 50, and the illuminating light is emitted to an object, such as an inspection site, in the patient 2 so as to form a light spot.

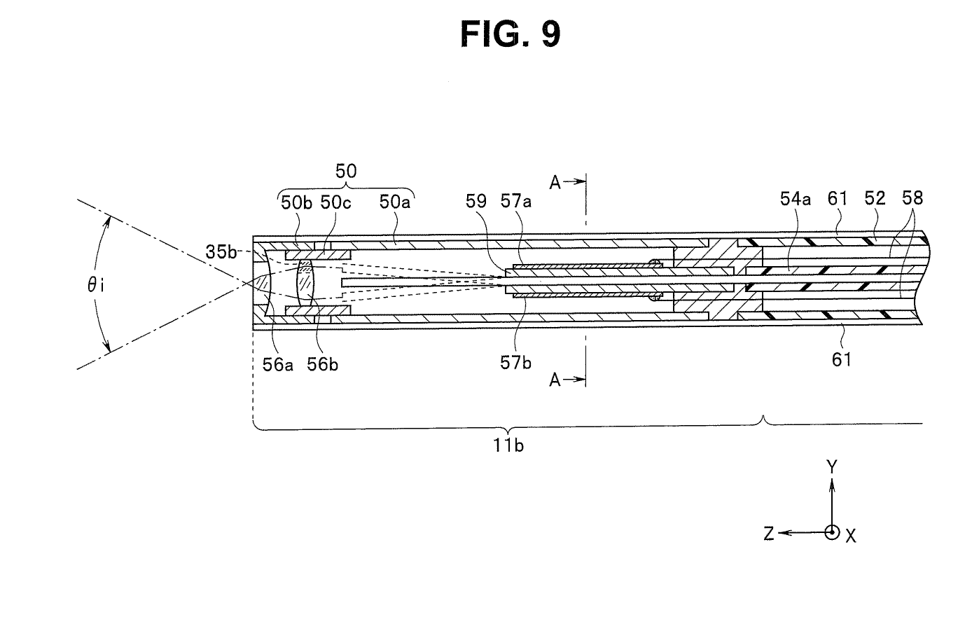

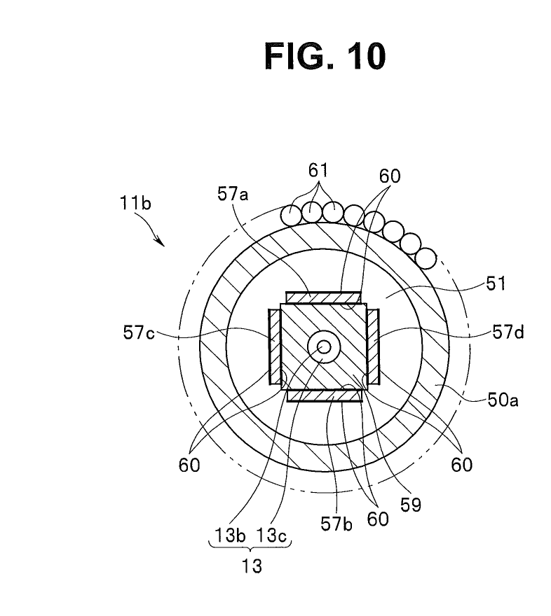

FIG. 9 shows a structure of a distal end side including the distal end portion 11b of the insertion portion 7b in FIG. 8. Note that an exterior tube 63 of FIG. 8 is not illustrated in FIG. 9 (and FIG. 10).

The cylindrical member 50 is simply illustrated in FIG. 8. In FIG. 9, the cylindrical member 50 includes: a cylindrical member body 50a; a first lens frame 50b holding a first lens 56a arranged near a distal end of the cylindrical member body 50a; and a second lens frame 50c engaged with a proximal end side of the first lens frame 50b, engaged with a distal end side of the cylindrical member body 50a, and holding a second lens 56b.

Instead of using the lens frames 50b and 50c shown in FIG. 9, the first lens 56a and the second lens 56b may be attached to the distal end of the cylindrical member 50 shown in FIG. 8.

The distal end side of the optical fiber 53 is arranged inside of the cylindrical member 50 (or the cylindrical member body 50a) forming the distal end portion 11b, along a substantially center axis of the cylindrical member 50.

The optical fiber 53 guides the illuminating light incident on the end surface on the proximal end side (incident side) and emits the light from the end surface on the distal end side (irradiation side).

At positions closer to the proximal end in the distal end portion 11b, piezoelectric elements 57a to 57d forming an actuator (or a scanner) 57 for swinging (vibrating) the distal end side of the optical fiber 53 in a direction orthogonal to the longitudinal direction of the optical fiber 53 are attached to an outer surface of a ferrule 59 as a connection member. FIG. 9 shows the piezoelectric elements 57a and 57b provided in the vertical direction, and FIG. 10 showing a cross section of a line A-A FIG. 9 shows the piezoelectric elements 57a, 57b, 57c, and 57d provided in the vertical and horizontal directions. FIG. 10 also shows that the optical fiber 53 includes a core 53b and a clad 53c.

The plate-shaped piezoelectric elements 57a to 57d forming the actuator 57 expand and contract in the longitudinal direction (Z axis direction in FIGS. 1 and 2) as a result of application of a drive signal from a drive unit 72 inside of the apparatus body 4B through drive lines 58 inserted into the insertion portion 7b.

The actuator 57 is provided with the piezoelectric elements 57a to 57d configured to vibrate the optical fiber 53, on vertical and horizontal outer surfaces in the ferrule 59 provided on an outer circumferential surface of the optical fiber 53.

Note that as can be understood from FIG. 10, the ferrule 59 is formed such that cross sections in a longitudinal direction (or an axial direction) and a perpendicular direction of the ferrule 59 are square, and the optical fiber 53 is inserted into a hole provided along a center axis of the ferrule 59 to hold the optical fiber 53.

As shown in FIG. 10, electrodes 60 in a flat plate shape are provided on both surfaces of the piezoelectric elements 57a to 57d, and the drive signal generated by the drive unit 72 can be applied to each of the electrodes 60 on each of both surfaces of the piezoelectric elements 57a to 57d through the drive lines 58.

A proximal end (back end) side of the ferrule 59 is held by the columnar holding member 51 for holding (fixing) the proximal end side of the ferrule 59.

Small diameter portions in which both ends in the longitudinal direction are cut out in a step shape are formed on an outer circumferential surface of the columnar holding member 51 as shown in FIG. 9, and a proximal end of the cylindrical member 50 and the distal end of the cylindrical tube 52 are fixed to respective small diameter portions. A flexible protection tube 54a covering the outer circumferential surface of the optical fiber 53 and protecting the optical fiber 53 is installed inside of the cylindrical tube 52.

As shown in FIGS. 9 and 10, a plurality of light receiving optical fibers 61 are arranged in a ring shape along outer circumferential surfaces of the cylindrical member 50 and the cylindrical tube 52, the light receiving optical fibers 61 serving as light receiving elements configured to receive the illuminating light reflected by the object. The light (return light or reflected light from the object) received by the light receiving optical fibers 61 is guided to a light receiving optical fiber 22b inside of the apparatus body 4B through an optical connection portion 62a of the connector 9b. Light (signal) emitted from an end surface of the light receiving optical fiber 22b is incident on a detection unit 73 and converted to an electrical signal. Note that the light (signal) emitted from proximal ends of the light receiving optical fibers 61 may be incident on the detection unit 73 without going through the light receiving optical fiber 22b.

The light receiving optical fibers 61 arranged in a ring shape are covered and protected by the flexible exterior tube 63 shown in FIG. 8.

Each of the scanning endoscopes 3B and 3C includes a memory 66 storing information, such as drive data for the actuator 57 to drive the distal end of the optical fiber 53 along a predetermined scan pattern and coordinate position data corresponding to irradiation positions when the distal end is driven. The information stored in the memory 66 is inputted to the controller 74 inside of the apparatus body 4B through a contact point of the connector 9b and a signal line and is stored in a memory 75.

Identification information indicating whether the optical member in the scanning endoscope 3B or 3C including the memory 66 is provided with a filter (for example, flag information indicating whether the fill is included or not included) is also stored in the memory 66. The controller 74 identifies or discriminates types of the scanning endoscopes 3B and 3C connected to the apparatus body 4B according to the identification information and performs control to generate different illuminating light according to the type of the connected scanning endoscope 3B or 3C. The controller 74 includes a discrimination circuit or a discrimination unit 74d (written as discrimination in FIG. 8) forming a discrimination section configured to identify or discriminate the type of the scanning endoscope 3B or 3C connected to the apparatus body 4B.

As shown in FIG. 8, the apparatus body 4B includes: the light source unit (or light source apparatus) 71 forming the illumination mechanism; the drive unit 72; the detection unit 73; the controller 74 configured to control each unit in the apparatus body 4B; and the memory 75 connected to the controller 74 and configured to store various pieces of information.

The light source unit 71 includes: an R light source 71a configured to generate light of a red wavelength band (also called R light); a G light source 71b configured to generate light of a green wavelength band (also called G light); a B light source 71c configured to generate light of a blue wavelength band (also called B light); and a multiplexer 71d configured to multiplex (mix) the R light, the G light, and the B light.

The R light source 71a, the G light source 71b, and the B light source 71c are formed by using, for example, laser light sources, and are configured to emit the R light, the G light, and the B light to the multiplexer 71d, respectively, when turned on by the control of the controller 74. The controller 74 includes a light source control section (or a light source control unit) 74a having a function of a control unit formed by a central processing unit (abbreviated as CPU) and the like configured to control discrete light emission of the R light source 71a, the G light source 71b, and the B light source 71c.

The light source control section 74a of the controller 74 sends pulsed control signals emitted at slightly different timings to the R light source 71a, the G light source 71b, and the B light source 71c, respectively. The R light source 71a, the G light source 71b, and the B light source 71c sequentially generate the R light, the G light, and the B light and emit the light to the multiplexer 71d.

The multiplexer 71d multiplexes the R light from the R light source 71a, the G light from the light source 71b, and the B light from the light source 71c and supplies the light to a light incident surface of the optical fiber 55b. The optical fiber 55b inputs the multiplexed R light, G light, and B light (also called RGB light) to the proximal end of the optical fiber 53. The optical fiber 53 guides the illuminating light incident on the proximal end and emits the guided light from the distal end surface as irradiating light.

The drive unit 72 includes a signal generator 72a, D/A converters 72b and 72c, and amplifiers 72d and 72e.

The signal generator 72a creates drive signals for swinging (or vibrating) the distal end of the optical fiber 53 and outputs the drive signals to the D/A converters 72b and 72c based on control by a scan control section 74b of the controller 74. The D/A converters 72b and 72c convert the digital drive signals outputted from the signal generator 72a into analog drive signals and output the signals to the amplifiers 72d and 72e, respectively.

The amplifiers 72d and 72e amplify the drive signals outputted from the D/A converters 72b and 72c, respectively, and output the created drive signals to the piezoelectric elements 57a to 57d as drive elements forming the actuator 57, through the drive lines 58.

The amplifier 72d generates drive signals for vibrating the piezoelectric elements 57a and 75b in a Y axis direction. On the other hand, the amplifier 72e generates drive signals for vibrating the piezoelectric elements 57c and 57d in an X axis direction.

FIG. 11 shows a waveform of the drive signal generated by the amplifier 72d. A horizontal axis in FIG. 11 denotes a time period t, and a vertical axis denotes a (alternating) voltage value of the drive signal. A peak voltage value temporally changes in the waveform. The drive signal of the amplifier 72e is for vibration in the X axis direction, obtained by shifting a phase of the drive signal shown in FIG. 11 by 90°.

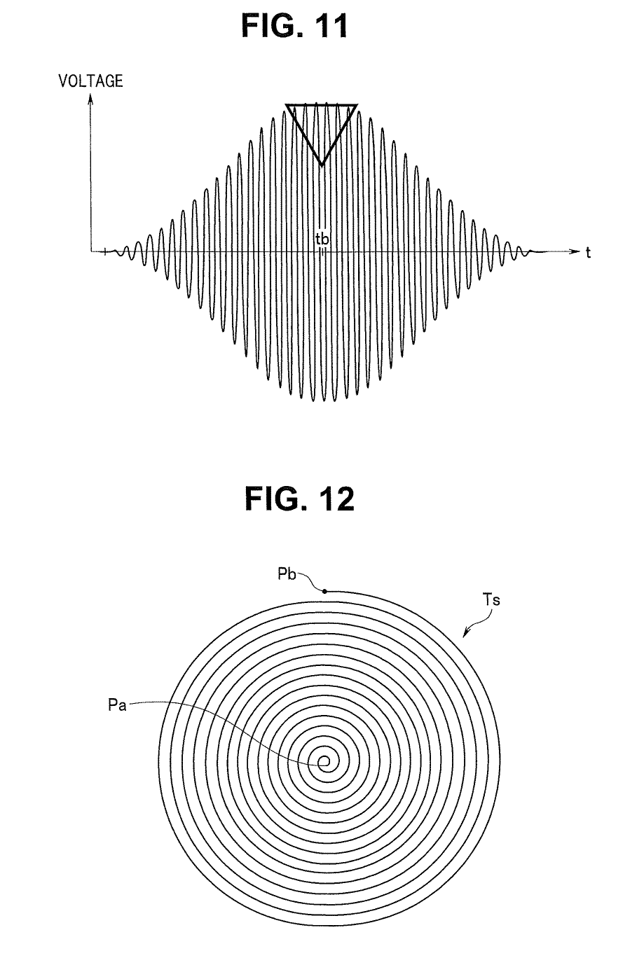

Therefore, the distal end of the optical fiber 53 is swung to form a trajectory Ts in a spiral shape as a predetermined scan trajectory as shown in FIG. 12. In FIG. 12, Pa denotes a scan start position (or a swing start position), which is at a position of a timing of a time period to in FIG. 11. A scan end position (or a swing end position) Pb in FIG. 12 is at a position of a timing of a time period tb in FIG. 11. The time period tb is a time period in which the voltage value of the drive signal for the vibration in the X axis direction is maximum, and the voltage value of the drive signal for the vibration in the Y axis direction is 0.

The pulsed illuminating light emitted along the trajectory Ts shown in FIG. 12 is applied in a spot shape to the object, and a scan range irradiated in a spiral shape on the object becomes the irradiation range.

FIG. 9 shows an illumination angle (or an irradiation angle) θi corresponding to the irradiation range of the illuminating light in the Y axis direction when the distal end of the optical fiber 53 is swung to form the trajectory Ts. In the present embodiment, the illumination angle can be approximated to be equal to the illumination angle θi in any radial direction as can be understood from the trajectory Ts shown in FIG. 12.

The irradiation lenses 56a and 56b as optical members shown in FIG. 9 are not provided with the filter 35b in the scanning endoscope 3B. However, in the scanning endoscope 3C, the irradiation lens 56b is provided with the filter 35b as indicated for example by a dotted line, at a position in the upper direction corresponding to the predetermined direction of the observation range (or the endoscopic image formed from the range). Note that the filter 35b may be provided on the irradiation lens 56a or may be provided on both of the irradiation lenses 56a and 56b.

The filter 35b is provided, for example, in a wedge shape as in the first embodiment, at the position corresponding to the upper direction of the endoscopic image. As shown in FIG. 9, the filter 35b is arranged at an upper position in an irradiation angle θiy in the vertical direction.

The filter 35b is set to, for example, the characteristic of the transmission characteristic C2a in FIG. 4. In the case of the characteristic, the filter 35b transmits light of only the red wavelength band in the incident illuminating light. The case is not limited to the case of the transmission characteristic C2a in FIG. 4. The characteristic of the transmission characteristic C2 may be set, or a different characteristic may be set.

In the scanning endoscope 3C provided with the filter 35b, the illumination characteristic of the irradiation range of the illuminating light emitted from the distal end of the optical fiber 53 varies between the case of the first illuminating light emitted through the part or region not provided with the filter 35b and the case of the second illuminating light emitted through the part or region provided with the filter 35b.

That is, the RGB light is emitted as the first illuminating light in the non-filter region, and the second illuminating light with only the R light is emitted in the filter region. In the observation from the outside of the patient 2, the upper direction of the distal end portion 11b can be figured out from the direction of the second irradiation range irradiated by the R light. Note that as described in the first embodiment, the shape of the filter 35b is not limited to the wedge shape.

The light receiving optical fibers 61 arranged in the ring shape and configured to receive the return light (of the illuminating light applied to the object) are set to have an observation view angle or an observation range based on an incident angle substantially narrower (or smaller) than the irradiation angle θiy.

As for the guiding characteristic of the light receiving optical fibers 61, optical fibers with a characteristic that does not substantially guide, to the incident surface, the incident light entering at an angle equal to or greater than a predetermined incident angle smaller than the irradiation angle θiy can be used. Alternatively, the image creation may be controlled to set the observation range from an observation view angle smaller than the irradiation angle θiy.

In this case, the light source control section 74a (control unit of the light source control section 74a) or the like controls the image creation section 74c so that the image creation section 74c creates an image from an optical signal received (detected) by the light receiving optical fibers 61 only in a period in which the illuminating light irradiates (scans) the irradiation range within the observation range (observation view angle of the observation range). In a period for irradiating (scanning) the outside of the observation range, the light source control section 74a or the like controls (can control) the image creation section 74c so that the image creation section 74c stops the action of creating the image from the optical signal received (detected) by the light receiving optical fibers 61.

As shown in FIG. 8, the detection unit 73 includes a detector 73a and an A/D converter 73b.

The detector 73a is formed by a photodiode or the like configured to receive R light, G light, and B light as return light emitted from a light emission end surface of a proximal end of a light receiving optical fiber 62b and photoelectrically convert the light. The detector 73a creates analog R, G, and B detection signals respectively corresponding to an intensity of the received R light, an intensity of the G light, and an intensity of the B light and outputs the R, G, and B detection signals to the A/D converter 73b.

The A/D converter 73b converts the analog R, G, and B detection signals sequentially inputted from the detector 73a into digital R, G, and B detection signals, respectively, and outputs the signals to the image creation section (or the image creation circuit) 74c forming a signal processing apparatus provided in the controller 74 and configured to generate an image (signal). The image creation section 74c outputs the created image signal to the monitor 6, and the monitor 6 displays the image of the image signal as an endoscopic image. Note that it may be defined that an image processing apparatus configured to create an image signal is formed by the detection unit 73 and the image creation section 74c.

The memory 75 stores in advance a control program and the like for controlling the apparatus body 4B. Information of coordinate positions read by the controller 74 of the apparatus body 4B from the memory 66 is also stored in the memory 75.

A CPU, an FPGA, or the like is used to form the controller 74, and the controller 74 reads the control program stored in the memory 75 to control the light source unit 71 and the drive unit 72 based on the read control program.

In the present embodiment, the second illumination mechanism as an illumination mechanism configured to emit the illuminating light for facilitating figuring out the predetermined direction in the irradiation range or the observation range (that is a range of part of the irradiation range) is also included in the case of the scanning endoscope 3B not including the filter 35b. The second illumination mechanism allows selecting, from a plurality of modes, the function of emitting the second illuminating light equivalent to the filter 35b.

The illumination mechanism according to the present embodiment includes: the light source unit 71 configured to generate illuminating light; the optical fiber 53 forming a light guiding portion configured to guide the illuminating light; the irradiation lens 56 (56a and 56b) forming an optical member configured to apply the illuminating light emitted from the distal end (surface) of the optical fiber 53 to the inside of the patient 2; and the light source control section 74a configured to control the light emission of the light source unit 71.

The user, such as a surgeon, can select one mode from a mode selection section (or a mode selection switch) 76 and input the selected mode signal to the controller 74. The light source control section 74a in the controller 74 controls the light source unit 71 to emit illuminating light including the first illuminating light and the second illuminating light in a mode corresponding to the mode signal.

When a first mode signal is selected, the light source control section 74a performs control to emit the second illuminating light for emission of light in the red wavelength region in a wedge shape, in substantially the same way as the filter 35b, for example.

When a second mode signal is selected, the light source control section 74a controls the light source unit 71 to emit the second illuminating light in a direction checking period different from the period for creating the endoscopic image. The action may be set based on the first mode signal in a normal action mode (mode is not selected), and the action may be set based on the second mode signal when the mode is selected.

Note that as described above, the predetermined scan range is scanned for substantially the same functions as the filter 35b in a first mode. On the other hand, unlike the first mode, a second mode is a mode in which the scan is performed to allow figuring out (visually checking), for example, the upper direction as the predetermined direction, and the light source unit 71 is caused to emit light in a scan period in the predetermined direction. Therefore, the mode selection section 76 can be interpreted as a selection switch for making a selection of generating third illuminating light similar to the function of the second illuminating light in the scan period for performing the scan in the predetermined direction in the second mode.

An illumination period for generating the illuminating light in the first mode may be defined as a first illumination period, and an illumination period for generating (emitting) the second illuminating light for checking the predetermined direction in the second mode may be defined as a second illumination period.

In the endoscope configured to pick up an image by the CCD as in the first embodiment, the illumination angle θil and the filter region are set so that the second irradiation range based on the filter region is formed (substantially) outside of the observation range. However, in the scanning endoscope of the present embodiment, the illumination may be performed in the predetermined direction in a mode different from the other directions in a range other than the scan range of the illuminating light for the image creation by the image creation section 74c.

The endoscope apparatus 1B of the present embodiment includes: the scanning endoscopes 3B and 3C as endoscopes including the insertion portion 7b with flexibility inserted into the paranasal sinus of the patient 2 forming the subject and capable of emitting the illumination light from the distal end of the insertion portion 7b toward the subject in the paranasal sinus; and the light source unit 71 forming an illumination mechanism configured to emit the illuminating light from the endoscope to the subject in the predetermined direction of the irradiation range of the illuminating light in a mode different from the other directions.

The endoscope apparatus 1B includes, as the mode, an illumination mechanism configured to emit the illuminating light in a state in which at least one of the light quantity and the wavelength band in the second illuminating light emitted in the predetermined direction is different from that of the first illuminating light emitted in the other directions.

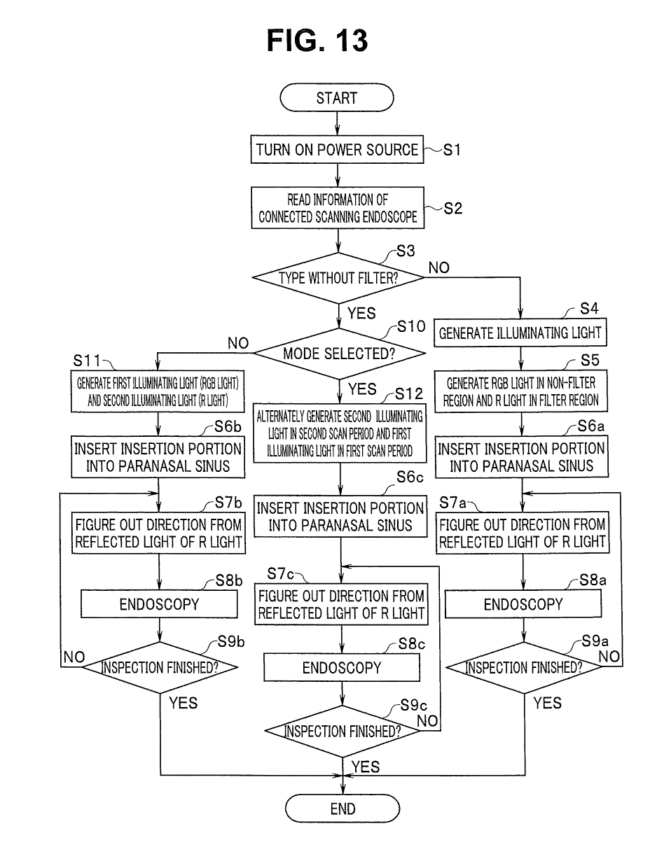

Next, an action of the present embodiment will be described. FIG. 13 shows a flowchart showing a process and the like of the present embodiment.

The surgeon connects the scanning endoscope 3B or 3C to the apparatus body 4B and turns on a power source switch of the apparatus body 4B as shown in step S1 of FIG. 13 to input a power source of the apparatus body 4B. The apparatus body 4B then enters an active state.

In the active state, the controller 74 in step S2 executes a process of reading the information of the type of the scanning endoscope connected to the apparatus body 4B from the memory 66 to discriminate the type of the connected scanning endoscope.

In step S3, the controller 74 discriminates whether the type of the connected scanning endoscope is the scanning endoscope 3B without the filter based on the stored identification information.

If the controller 74 discriminates that the filter is included (that is, the scanning endoscope 3C) in the discrimination process of step S3, the light source control section 74a of the controller 74 controls the light source unit 71 to generate normal illuminating light in step S4. The light source control section 74a applies the drive signal to the piezoelectric elements 57a to 57d, and the distal end of the optical fiber 53 swings to depict the trajectory Ts shown in FIG. 12.

As shown in step S5, in the illuminating light emitted from the distal end of the optical fiber 53, the illuminating light passing through the non-filter region in the irradiation lenses 56a and 56b becomes the RGB light (first illuminating light), and the illuminating light passing through the filter region becomes the R light (second illuminating light). The irradiation range corresponding to the trajectory Ts of FIG. 12 in the object is illuminated.

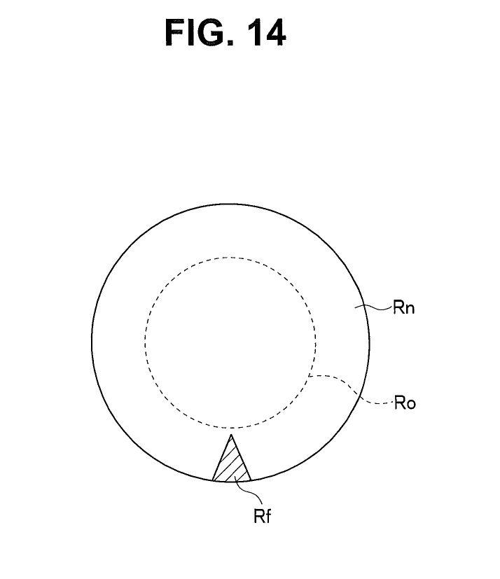

FIG. 14 shows the irradiation range in which the illuminating light is applied in step S5. As shown in FIG. 14, the second region based on the R light (second illuminating light) passing through the filter region is a region in a wedge shape as indicated by oblique lines, and the remaining substantially circular region indicates the first region based on the RGB light (first illuminating light) passing through the non-filter region. In FIG. 14, the second region (as the second irradiation range) based on the filter region is indicated by Rf, and the first region (as the first irradiation range) based on the non-filter region is indicated by Rn. As shown in FIG. 9, the illuminating light incident on the upper part (above the optical axis) of the irradiation lens 56 is emitted below the optical axis of the irradiation lens 56. Therefore, FIG. 14 shows an example in which the second region Rf based on the filter region on the upper side is formed in the lower direction.

In FIG. 14, a dotted line shows an observation range Ro. The observation range Ro is set to be inside of the second region Rf.

Therefore, when an image of the observation range Ro is formed to display an endoscopic image on the monitor 6, the second region Rf does not appear in the endoscopic image. As described, the light source control section 74a controls the image creation section 74c to generate an image from the optical signal received by the light receiving optical fibers 61 in the period in which the illuminating light scans inside of the observation range Ro and controls the image creation section 74c not to create an image outside of the period, for example.

The surgeon checks the irradiation state in which the second region Rf is formed, and the surgeon inserts the insertion portion 7b into the maxillary sinus 41 in the paranasal sinus 2a of the patient 2 as shown in step S6a. Note that the surgeon often performs operation of rotating the insertion portion 7b about the longitudinal direction of the insertion portion 7b to smoothly perform the operation of inserting the insertion portion 7b. Therefore, in the state that the insertion operation is performed, the surgeon cannot figure out the actual direction of the upper direction of the endoscopic image.

As shown in step S7a, the surgeon can observe the reflected light from the irradiation range emitted to the inner wall of the maxillary sinus 41 from the outside of the patient 2 to figure out the direction of the irradiation range based on the R light (second illuminating light), that is, the upper direction of the endoscopic image.

Note that the situation in which the illuminating light is emitted to the inner wall of the maxillary sinus 41 is substantially the same state as the situation of irradiation as shown in FIG. 6 in the first embodiment. The observation range using the light receiving optical fibers 61 in this case is also formed inside of the irradiation range using the optical fiber 53 as in the case shown in FIG. 6.

The surgeon can figure out the direction of the second region based on the R light (second illuminating light) to smoothly perform the operation of moving the distal end portion 11b from the currently observed site toward a site to be observed (inspected) next. The surgeon then performs endoscopy or the like of the site to be inspected as shown in step S8a.

In next step S9a, the controller 74 judges whether the surgeon has performed an instruction operation for ending the inspection. If the instruction operation for ending the inspection is not performed, the process returns to step S6a, and the same process or the like is repeated. If the instruction operation for ending the inspection is performed, the process of FIG. 12 ends.

On the other hand, if the controller 74 discriminates that the filter is not included in step S3, the controller 74 (the light source control section 74a of the controller 74) further judges whether the mode selection is performed in step S10. If a judgement result indicates that the mode selection is not performed, the controller 74 (the light source control section 74a of the controller 74) performs a control action in the first mode as described in next step S11 and subsequent steps.

In step S11, the light source control section 74a controls the light source unit 71 to generate the first illuminating light (RGB light) and the second illuminating light (R light) as in the case in which the filter 35b is provided on the irradiation lens 56b.

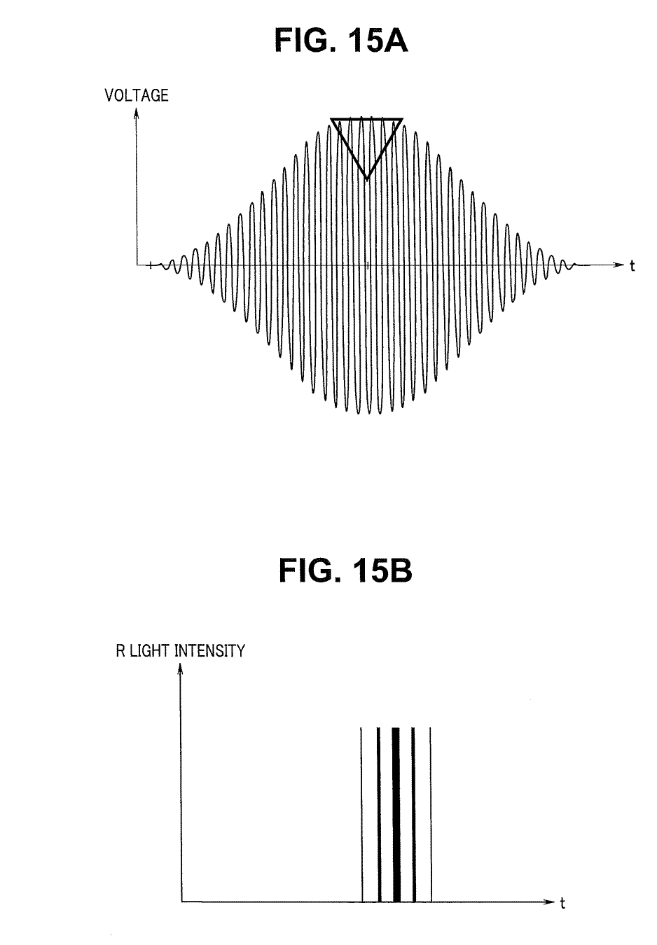

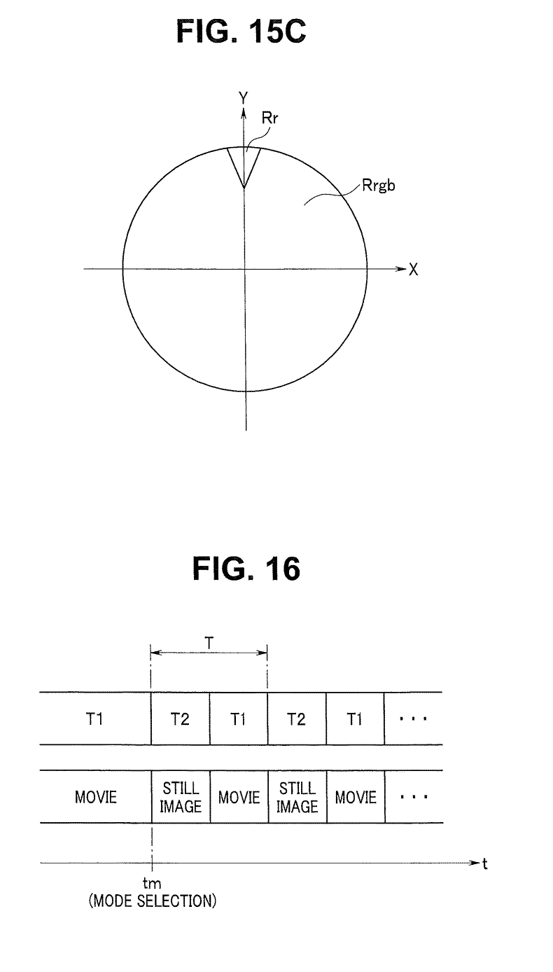

More specifically, in the drive signal in the Y axis direction as shown in FIG. 15A, the light source control section 74a controls the light source unit 71 to generate only the R light as shown in FIG. 15B in periods equivalent to the region in the wedge shape for generating the illuminating light for checking the direction.

In the drive signal shown in FIG. 15A, the light source control section 74a performs the control to generate the R light as shown in FIG. 15B only in the periods for scanning the region in the wedge shape. In FIG. 15B, the larger the width, the longer the period for generating the R light is. Note that FIG. 15B shows only the periods for generating only the R light as the second illuminating light. The RGB light is generated in periods other than the periods shown in FIG. 15B (indicated by vertical lines). However, pulsed R light, G light, and B light are actually cyclically emitted.

As shown in FIG. 15C, the light source unit 71 generates the first illuminating light (RGB light) and the second illuminating light (R light) corresponding to substantially the same wedge shape as in the case provided with the filter region and emits the light to the optical fiber 53. The second illuminating light as illuminating light of the R light is generated in the region in the wedge shape as shown in FIG. 15C according to the drive signal of FIG. 15A and the timing of the generation of the R light in FIG. 15B, and the remaining region is the first illuminating light that is the RGB light. The illuminating light of FIG. 15C is emitted toward the object through the irradiation lenses 56a and 56b including only the non-filter region, and an irradiation range corresponding to FIG. 15C is formed.

The surgeon can check that the irradiation range corresponding to FIG. 15C is formed on the object. In FIG. 15C, the region of the R light is indicated by Rr, and the region of the RGB light is indicated by Rrgb. Note that when the scanning endoscope 3B is set to the same state as in FIG. 9, the region Rr of the R light is formed on the lower side in the Y axis direction on the object side. The irradiation range in the state of irradiation on the object side is the same as in the case of FIG. 14.

As can be understood from FIGS. 15C and 14, the illumination in the first mode functions in the same way as when the filter 35b is provided.

After checking the irradiation state, the surgeon inserts the insertion portion 7b into the maxillary sinus 41 in the paranasal sinus 2a of the patient 2 as shown in step S6b.

As shown in step S7b, the surgeon can observe the reflected light from the irradiation range emitted to the inner wall of the maxillary sinus 41 from the outside of the patient 2 to figure out the direction of the irradiation range based on the R light (second illuminating light), that is, the upper direction of the endoscopic image.

The surgeon can figure out the direction of the irradiation range based on the R light (second illuminating light) to smoothly perform the operation of moving the distal end portion 11b from the currently observed site toward the site to be observed (inspected) next. The surgeon then performs endoscopy or the like of the site to be inspected as shown in step S8b.

In next step S9b, the controller 74 judges whether the surgeon has performed an instruction operation for ending the inspection. If the instruction operation for ending the inspection is not performed, the process returns to step S6b, and the same process or the like is repeated. If the instruction operation for ending the inspection is performed, the process of FIG. 12 ends.

If the mode selection of step S10 is performed, the controller 74 (the light source control section 74a of the controller 74) controls the light source unit 71 to perform illumination in the second mode different from the first mode in step S12. As described below, the light source control section 74a controls the light source unit 71 to generate the second illuminating light in a second scan period (second illumination period) and (alternately) generate the first illuminating light in a first scan period (first illumination period).

In this case, the light source control section 74a controls the light source unit 71 to generate the illuminating light for checking the direction when the filter 35b is provided or in the scan period (or illumination period) for checking the direction different from the normal scan period (or illumination period) in the first mode. FIG. 16 shows normal scan periods T1 and scan periods T2 for checking the direction. As shown in FIG. 16, when the second mode is not selected, the controller 74 controls the light source unit 71, the drive unit 72, the detection unit 73, and the like to operate in the normal scan periods T1.

When the second mode is selected, the scan period T2 for checking the direction and the normal scan period T1 are repeated at a predetermined cycle T. In this state, when an operation of stopping the second mode is further performed, an action of the normal scan period T1 is performed. The surgeon can also select an action in the normal scan period T1.

That is, the surgeon can select, as the action in the normal scan period T1, to perform the scan and the illumination as in the first mode or to perform the scan and the illumination in the case where the scanning endoscope 3C provided with the filter 35b is connected.

As shown in FIG. 16, in the scan period T2 for checking the direction, the endoscopic image of a final frame period in the normal scan period T1 just before the scan period T2 for checking the direction may be displayed as a still image (movie in the scan period T1). For example, the light source control section 74a may control the action of the image creation section 74c to output, to the monitor 6, the endoscopic image of the final frame period in the scan period T1 as an image signal of a still image in the scan period T2.

In this case, when the scan periods T1 and T2 are set to about 1/30 seconds to 1/10 seconds for example, the surgeon can observe the endoscopic image like a movie with slightly fewer frames than movement of a normal movie.

The illuminating light in the scan period T2 for checking the direction can be visually checked from the outside of the patient 2 to figure out the upper direction in the endoscopic image of the observation range.

Note that other than the case of alternately performing the scan periods T1 and T2, only the scan and the illumination for checking the direction may be continued when the second mode is selected, until the operation for stopping the second mode is subsequently performed.

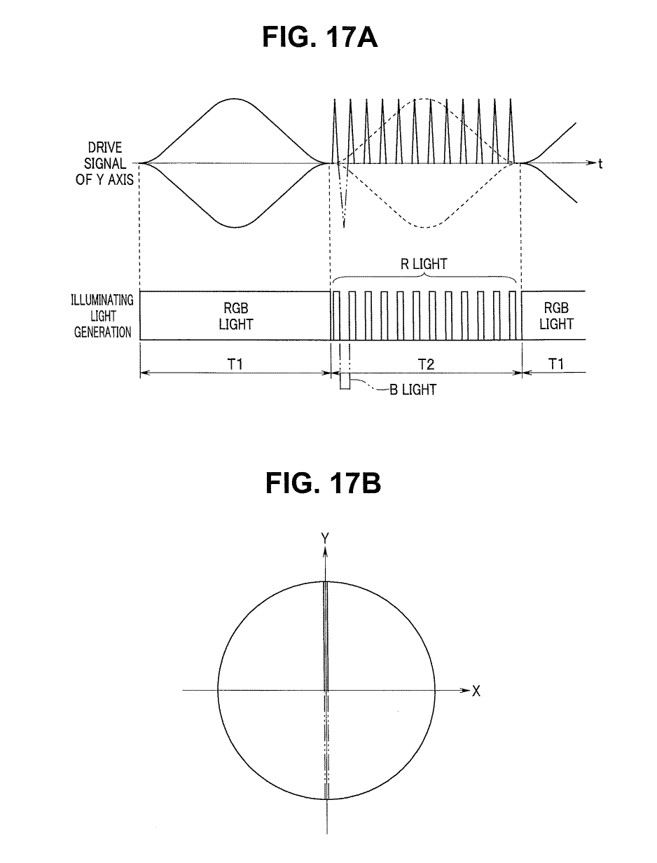

FIG. 17A shows a drive signal of the Y axis (direction) and a period for generating the illuminating light. In the scan period T1, the drive signal is outputted in the Y axis direction and the X axis direction, and the light source unit 71 generates the RGB light that is the first illuminating light. Note that although the drive signal in the scan period T1 is indicated by a waveform with only a contour in FIG. 17A, the waveform of the drive signal is actually indicated as shown in FIG. 11.

On the other hand, in the scan period T2, the drive signal in the (positive) Y axis direction as the predetermined direction is outputted, and the light source unit 71 generates only the R light that is the second illuminating light only in the period in which the drive signal in the Y axis direction is outputted (period in which the drive signal is positive in the Y axis direction). Although the case of generating the R light will be described, light of the G light or the like (different from the RGB light) may be generated instead of the R light.

In this way, only the second illuminating light is generated in the scan period T2. The first illuminating light and the second illuminating light are emitted to the optical fiber 53. As shown in FIG. 17A, the pulsed R light is generated in a plurality of scan periods in the positive Y axis direction. FIG. 17B shows, in a coordinate system at the position of the distal end of the optical fiber 53, that the R light is outputted to the optical fiber 53 only in the period in which the drive signal is outputted in the positive Y axis direction.

In the scan period T2, the irradiation range of the R light corresponding to FIG. 17B is formed on the object through the irradiation lenses 56a and 56b including only the non-filter region. The surgeon can figure out the upper direction from the irradiation range corresponding to FIG. 17B.

Note that to further facilitate checking (figuring out) the upper direction as the predetermined direction in which the second illuminating light is emitted, the R light may be generated at the timing of the upper direction, and for example, the B light different from the R light (and different from the RGB light) may be further generated at the timing of the lower direction that is a direction on the opposite side of the upper direction.

For example, a drive signal may also be generated in a negative Y axis direction as indicated by an alternate long and two short dashes line in FIG. 17A, and the light source control section 74a may generate the B light as indicated by an alternate long and two short dashes line in a period in which the drive signal is generated. In this case, the B light is outputted from the light source unit 71 at a timing of the lower direction as indicated by an alternate long and two short dashes line in FIG. 17B. Note that although FIG. 17A shows an example of generating the B light only once for the simplification, generating the B light for a plurality of times as in the case of the R light is actually desirable.

The surgeon can easily figure out that the R light indicates the upper direction and the B light indicates the lower direction, from the reflected light when the second illuminating light is emitted to the object.

After checking the situation of the irradiation corresponding to FIG. 17B, the surgeon inserts the insertion portion 7b into the maxillary sinus 41 in the paranasal sinus 2a of the patient 2 as shown in step S6c in FIG. 13.

As shown in next step S7c, the surgeon can observe the reflected light from the irradiation range applied to the inner wall of the maxillary sinus 41 from the outside of the patient 2 to figure out the direction of the irradiation range based on the R light (second illuminating light), that is, the upper direction of the endoscopic image.

The surgeon can figure out the direction of the irradiation range based on the R light (second illuminating light) to smoothly perform the operation of moving the distal end portion 11b from the currently observed site toward the site to be observed next.

In next step S8c, the controller 74 judges whether the surgeon has performed an instruction operation for ending the inspection. If the instruction operation for ending the inspection is not performed, the process returns to step S6c, and the same process or the like is repeated. If the instruction operation for ending the inspection is performed, the process of FIG. 12 ends.

According to the present embodiment operated in this way, a predetermined direction, such as an upper direction, of the endoscopic image can be figured out from the outside of the patient 2 not only when the filter 35b is provided on the optical member, but also when the scanning endoscope 3B not provided with the filter 35b on the optical member is used.

Therefore, according to the present embodiment, the endoscope apparatus 1B capable of smoothly performing an inspection inside of the paranasal sinus 2a, a treatment using a treatment instrument, and the like can be provided.

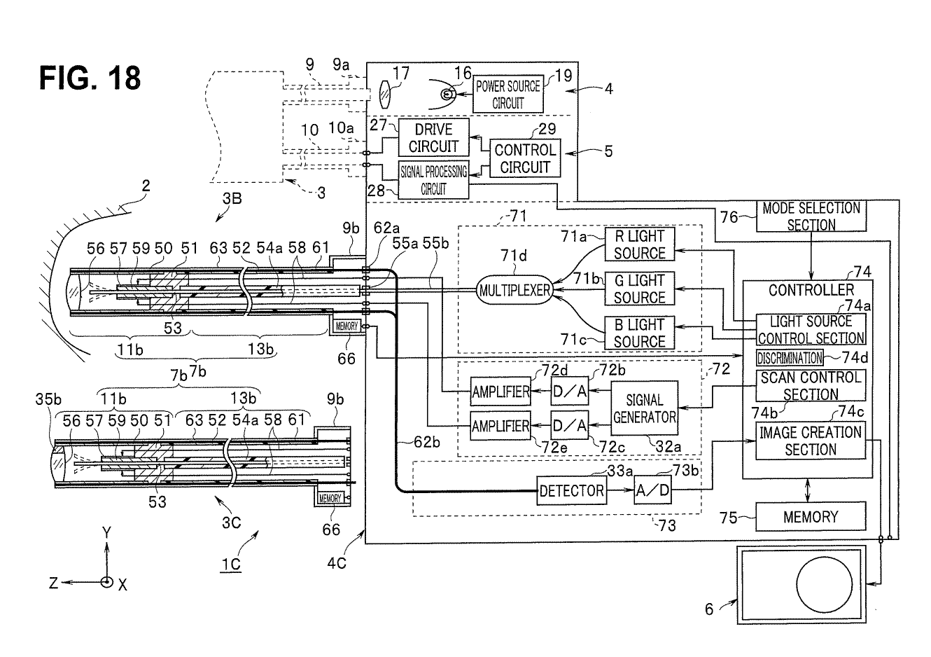

Although the endoscope apparatus 1B using the scanning endoscopes 3B and 3C is described in the second embodiment described above, an endoscope apparatus 1C of a modification may be formed as shown in FIG. 18. The endoscope apparatus 1C further has a configuration that allows connecting and using the endoscope 3 including the image pickup device shown in FIG. 1 in the endoscope apparatus 1B of FIG. 8. That is, the endoscope apparatus 1C includes the endoscope 3 and an apparatus body 4C that allows connecting and using an arbitrary one of the two types of scanning endoscopes 3B and 3C. Note that FIG. 18 shows a case in which the scanning endoscope 3B is connected to the apparatus body 4C as in the case of FIG. 8.

The apparatus body 4C includes the apparatus body 4B shown in FIG. 8, the light source apparatus (or the light source unit) 4 of FIG. 1, and the video processor 5. The endoscope 3, the scanning endoscopes 3B and 3C, the apparatus body 4B, the light source apparatus 4, and the video processor 5 are already described and will not be written (described) here.

In the present modification, the action is as described in the first embodiment when the endoscope 3 is connected to the light source apparatus 4 and the video processor 5 in the apparatus body 4C as indicated by dotted lines. The action in this case is already described in the first embodiment, and the description will not be repeated. The action is as described in the second embodiment when the scanning endoscope 3B or 3C is connected to the apparatus body 4C. The action in this case is already described in the second embodiment, and the description will not be repeated.

Note that part of the embodiments and part of the modification may be partially combined.

The content in the original claims may be changed within the range disclosed in the specification and the drawings.

What is claimed is:

1. An endoscope apparatus comprising:
an endoscope comprising an insertion portion with flexibility inserted into paranasal sinus of a subject and capable of emitting illuminating light from a distal end of the insertion portion toward the subject inside of the paranasal sinus; and
an illumination mechanism configured to emit the illuminating light from the endoscope to the subject in a predetermined direction of an irradiation range of the illuminating light in a mode different from other directions.

2. The endoscope apparatus according to claim 1, wherein
the illumination mechanism emits first illuminating light in the other directions and emits second illuminating light in which at least one of a light quantity and a wavelength band is different from the first illuminating light in the predetermined direction.

3. The endoscope apparatus according to claim 2, further comprising
an image creation section configured to create an observation image in a range of part of an illumination range of the illuminating light in the object, wherein
the illumination mechanism emits the illuminating light in the predetermined direction of the illumination range of the illuminating light other than the part of the range where the observation image is created by the image creation section, in the mode different from the other directions.

4. The endoscope apparatus according to claim 2, wherein
the illumination mechanism comprises an illumination window provided at the distal end of the insertion portion and configured to emit the first illuminating light and the second illuminating light within a predetermined illumination angle and
further comprises an observation window provided adjacent to the illuminating window at the distal end of the insertion portion and configured to receive only incident light within an observation view angle smaller than the illuminating angle and equivalent to an observation range that is part of the illumination range, and
the illumination mechanism emits the second illuminating light to the subject within a range greater than the observation view angle and equal to or smaller than the illumination angle.

5. The endoscope apparatus according to claim 2, wherein
the illumination mechanism emits the first illuminating light and the second illuminating light in the mode as a state that allows figuring out the predetermined direction in which a second region in the irradiation range exists, through visual check of the second region in the irradiation range where the second illuminating light is emitted or visual check of only a first region in the irradiation range where the first illuminating light is emitted, from outside of the subject.

6. The endoscope apparatus according to claim 1, wherein
the illumination mechanism comprises a light guiding portion arranged on the endoscope and provided with a plurality of fibers in a bundle configured to guide the illuminating light to the distal end of the insertion portion, and
in the light guiding portion, a light guiding characteristic of the fibers corresponding to the predetermined direction in the irradiation range of the illuminating light and a light guiding characteristic of the fibers corresponding to the other directions are different.

7. The endoscope apparatus according to claim 1, wherein
the illumination mechanism comprises:
an optical member provided at the distal end of the endoscope and configured to emit the illuminating light to the subject; and
a filter provided on the optical member and provided on an optical path of the illuminating light emitted in the predetermined direction.

8. The endoscope apparatus according to claim 1, wherein
the endoscope is a scanning endoscope configured to scan the illuminating light from the distal end of the insertion portion toward the subject, and
the illumination mechanism comprises:
a light source unit capable of emitting light in different colors as the illuminating light emitted from the distal end of the insertion portion; and
a control unit configured to control the light source unit to emit light in a color different from a case in which the illuminating light is emitted in a direction other than the predetermined direction when the illuminating light is emitted in the predetermined direction in a scan range forming the irradiation range in which the illuminating light is applied in the scanning endoscope.

9. The endoscope apparatus according to claim 8, wherein
the illumination mechanism emits the illuminating light to the subject, to a region along the predetermined direction.

10. The endoscope apparatus according to claim 2, further comprising:
a light source unit configured to generate the illuminating light;
a light guiding portion provided on the endoscope and comprising at least one optical fiber configured to guide the illuminating light generated by the light source unit to the distal end of the insertion portion;
an optical member arranged to oppose the distal end of the light guiding portion and configured to emit the illuminating light emitted from the distal end of the light guiding portion toward the subject;
a light receiving device provided on the endoscope and configured to receive return light from an observation range that is part of a range of the irradiation range in the illuminating light emitted to the subject; and
an image processing apparatus configured to create an image signal corresponding to the observation range based on an output signal outputted from the light receiving device or an emitted optical signal and output the created image signal to a display apparatus.

11. The endoscope apparatus according to claim 10, wherein
the endoscope comprises, as the light receiving device: an objective lens configured to form an optical image of the observation range; and an image pickup device arranged at an image forming position of the objective lens and configured to photoelectrically convert the optical image to a two-dimensional image.

12. The endoscope apparatus according to claim 10, comprising
a scanning endoscope as the endoscope, the scanning endoscope comprising: an actuator configured to drive a distal end of the light guiding portion to depict a predetermined scan trajectory; and an optical member arranged to oppose the distal end of the light guiding portion and configured to emit the illuminating light emitted from the distal end of the light guiding portion to scan, with a light spot, the irradiation range that is a predetermined scan range in the subject, wherein the light receiving device is formed by a light receiving optical fiber.

13. The endoscope apparatus according to claim 12, wherein
a first type of a scanning endoscope in which the optical member provided with a filter with a transmission characteristic for generating the second illuminating light can be detachably connected to the light source unit, and a second type of a scanning endoscope in which the optical member not provided with the filter can be detachably connected to the light source unit.

14. The endoscope apparatus according to claim 13, further comprising
a control unit configured to control the light source unit to cause the illuminating light generated by the light source unit to enter the optical member provided with the filter through the light guiding portion to thereby emit the first illuminating light and the second illuminating light generated by the optical member to the irradiation range when the first type of the scanning endoscope is connected to the light source unit and
configured to control the light source unit to generate the first illuminating light and the second illuminating light in the light source unit as the illuminating light entering the optical member not provided with the filter through the light guiding portion when the second type of the scanning endoscope is connected to the light source unit.

15. The endoscope apparatus according to claim 13, further comprising
a discrimination unit configured to discriminate whether the scanning endoscope connected to the light source unit is the first type or the second type.

16. The endoscope apparatus according to claim 15, comprising
a control unit configured to control the light source unit to cause the illuminating light generated in the light source unit to enter the optical member provided with the filter through the light guiding portion when a discrimination result of the discrimination unit indicates that the first type of the scanning endoscope is connected and
configured to control the light source unit to generate the first illuminating light and the second illuminating light as the illuminating light generated by the light source unit when a discrimination result of the discrimination unit indicates that the second type of the scanning endoscope is connected.

17. The endoscope apparatus according to claim 13, wherein
the control unit controls the light source unit to emit pulsed light of each of red, green, and blue as the first illuminating light in a first scan period for scanning the observation range with the light spot when the second type of the scanning endoscope is connected to the light source unit and
controls the light source unit to emit pulsed light of one of red, green, and blue as the second illuminating light different from the first illuminating light at a timing indicating the predetermined direction in a second scan period for scanning outside of the observation range with the light spot.

18. The endoscope apparatus according to claim 13, further comprising
a selection switch for making a selection for generating third illuminating light indicating a predetermined direction of the observation range in the irradiation range including the observation range.

19. The endoscope apparatus according to claim 18, wherein
when the second type of the scanning endoscope is connected to the light source unit, and the selection is made through the selection switch, the control unit controls and drives the actuator to scan the light spot in the predetermined direction in the observation range and controls the light source unit to emit third illuminating light in a third scan period in which the light spot is scanned in the predetermined direction, and
the control unit further controls the image processing apparatus to stop creating the image signal in the third scan period.

20. The endoscope apparatus according to claim 19, wherein
in the third scan period in which the image processing apparatus stops generating the image signal, the control unit controls the image processing apparatus to output, to the display apparatus, a still image that is an image of the image signal created by the image processing apparatus just before the third scan period.
